(19) United States
(12) Patent Application Publication
(10) Pub. No.: US 2007/0259948 A1
(43) Pub. Date: Nov. 8, 2007

(54) Compounds with antifungal properties and process thereof

(76) Inventors: Mukund Keshav Gurjar, Pune (IN); Radhika Dilip Wakharkar, Pune (IN); Hanumant Bapurao Borate, Pune (IN); Ramesh Ganesh Kelkar, Pune (IN); Andiappan Murugan, Pune (IN); Mohan Anand Chandavarkar, Mumbai (IN); Shreerang Vidyadhar Joshi, Mumbai (IN); Sharangi Ravindra Vaiude, Mumbai (IN)

Correspondence Address: BLANK ROME LLP, 600 NEW HAMPSHIRE AVENUE, N.W., WASHINGTON, DC, 20037, US

(21) Appl. No.: 11/498,805

(22) Filed: Aug. 4, 2006

(30) Foreign Application Priority Data
May 8, 2006 (IN) 713/MUM/2006

(51) Int. Cl. A61K 31/381; A61K 31/341; C07D 307/26; C07D 317/00; C07D 409/02

(52) U.S. Cl. 514/444; 514/473; 549/323; 549/453; 549/60

(57) ABSTRACT

The present invention relates to compounds of formula (1), its solvates and pharmaceutically acceptable salts having antifungal activity and its pharmaceutical composition comprising an effective amount of compound of formula (1)

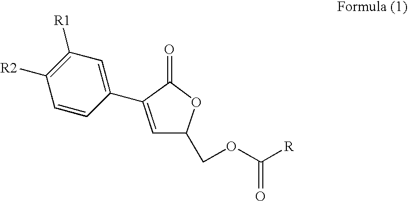

wherein R is substituted alkyl, alkenyl, aryl, heteroaryl, 2-thienyl, 3-thienyl, halothienyl, haloalkyl, halophenyl, or pyrrolyl; and R₁ and R₂, each independent of the other, are hydrogen, halogen, or alkoxy. The invention also relates to a process for the preparation of said compounds by contacting the intermediate alcohol, prepared from 1,2-O-isopropylideneglyceraldehyde and substituted phenylacetates, with acid chlorides under appropriate conditions to obtain some of the preferred compounds of the invention.

### FIELD ON INVENTION

The present invention relates to compounds, their solvates and pharmaceutically acceptable salts having antifungal activity. The invention also relates to a process for the preparation of said compounds and their pharmaceutical compositions.

### BACKGROUND OF THE INVENTION

5-Aceloxymethyl-3-aryl-2H,5H-furan-2-one based antifungal agents are known to exhibit anti-fungal activity against various strains of fungi. However, occurance of fungal infections in larger number and emergence of pathogens resistant to some of the antifungal drugs already existing in the market, makes necessary the development of broad spectrum antifungal agents which are effective against certain mycoses (such as aspergillosis) for which no effective treatment is available.

In the past decades the frequency and types of life-threatening fungal infections have increased dramatically in immuno-compromised patients. Several factors have contributed to the rise such as the expansion of severely ill and or immuno-compromised patient populations with HIV infection, with chemotherapy induced neutropenia, and receiving immunosuppressive therapy; more invasive medical procedures, such as extensive surgery and the use of prosthetic devices and vascular catheters; treatment with broad-spectrum antibiotics or glucocorticosteroids; and peritoneal dialysis or hemodialysis.

This problem of increased fungal infections is accentuated by the emergence of fungal strains which are resistant to currently used antifungal agents. Major opportunistic fungal pathogens include Candida albicans, Aspergillus, Fusurian spp. Other species of Candida such as C, krusei, C tropicalis, C.glabrata are major causative agents of candidiasis. Invasive pulmonary aspergillosis is a leading cause of mortality in bone marrow transplant recipients. HIV-infected patients are particularly susceptible to mucosal candidiasis, cryptcoccal meningitis.

Fluconazole is the preferred broad spectrum anti-fungal agent used in treatment of fungal infections. In recent times resistance of Candida albicans the most common cause of mucosal candidiasis in HIV-infected patients, after long-term suppressive therapy, to azoles, particularly fluconazole, is a cause of increasing concern. Resistance to fluconazole in other Candida species and in Cryptococcus neoformans has also been reported. Also, fluconazole appears to be less active against the two emerging Candida species, C. glabratta and C. krusei. Infection with Aspergillus, although not common, is frequently life-threatening and fluconazole has only moderate activity against this fungus. This has necessitated the need for new antifungal agents with broad spectrum of antifungal activity, which this invention seeks to provide.

Pour et al., J. Med. Chem. 44:2701-2706, 2001 discloses antifungal agents having chemical formulas similar to formula (1) of the present invention wherein R is p-ClC₆H₄, CH₃ or (CH₃)₃C.

### SUMMARY OF THE INVENTION

An object of the present invention is to provide compounds and methods of making compounds of formula (1) as an antifungal agent

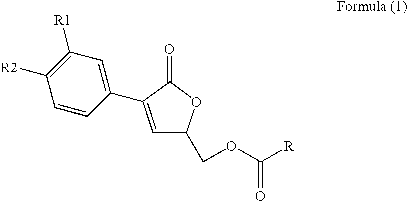

wherein R is substituted alkyl, alkenyl, aryl, heteroaryl, 2-thienyl, 3-thienyl, halothienyl, haloalkyl, halophenyl, or pyrrolyl; and R₁ and R₂, each independent of the other, is hydrogen, halogen, or alkoxy.

Another object of the present invention is to provide solvate or pharmaceutically acceptable salts of compounds of formula (1).

Another object of the present invention is to provide an antifungal agent having broader spectrum of activity.

Another object of the present invention is to provide a process for the preparation of antifungal compound of formula (1).

Another object of the present invention is to provide a pharmaceutical composition comprising an effective amount of antifungal compound of formula (1).

The present invention relates to antifungal compounds represented by general formula (1), its solvate or pharmaceutically acceptable salts and its pharmaceutical composition. The invention also relates to a process for the preparation of compounds of formula (1). In the process of preparation of compounds of formula (1) the compounds may also be obtained as its solvates or pharmaceutically acceptable salts.

The advantages of the compound of the present invention includes: 1) broad spectrum anitifungal activity; 2) improved activity against candida strains resistant to known azoles; 3) activity against aspergillus and other emerging fungal pathogens; and 4) improved safety profile than earlier azoles while retaining its broad spectrum of activity.

### DETAILED DESCRIPTION OF THE PREFERRED EMBODIMENTS

In accordance with the object, the present invention provides compounds and methods for making compounds of formula (1), its solvates or pharmaceutically acceptable salts

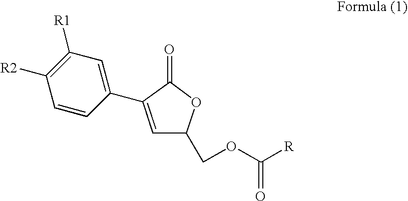

wherein R is substituted alkyl, alkenyl, aryl, heteroaryl, 2-thienyl, 3-thienyl, halothienyl, haloalkyl, halophenyl, or pyrrolyl; and R₁ and R₂, each independent of the other, are hydrogen, halogen, or alkoxy.

In one embodiment, the present invention is directed to compounds of formula (1) wherein R is substituted alkyl, alkenyl, aryl, heteroaryl, 2-thienyl, 3-thienyl, halothienyl, haloalkyl, halophenyl, or pyrrolyl; and R₁ and R₂, each independent of the other, is hydrogen, halogen, or alkoxy; with the proviso that R cannot be p-ClC₆H₄, CH₃ or (CH₃)₃C, when a) R₁ is H and R₂ is methoxy (OCH₃), b) R₁ and R₂ are both Cl, c) R₁ is Cl and R₂ is H, or d) R₁ is H and R₂ is Br.

In an other embodiment of the invention, R is preferably phenyl, allyl, 2-thienyl, 3-thienyl, 4,5-dibromo-2-thienyl, 2-chloroethyl, 3-chloropropyl, 4-chlorobutyl, 3-iodophenyl, or 2-pyrrolyl; and RI and R₂ are, independently, H, Br, Cl, F, or methoxy. In the most preferred compounds of formula (1), R is 2- or 3-thienyl, when R₁ is Br and R₂ is H or when R₁ and R₂ are Cl; or R is 2-chloroethyl or 3-chloropropyl, when R₁ is F and R₂ is H, or R₁ and R₂ are Cl.

Another embodiment provides pharmaceutical composition comprising a compound of formula (1) or its solvates or pharmaceutically acceptable salts of such compound having at least one salt forming group together with a pharmaceutical carrier, such as water, alcohol, urea, or propylene glycol.

Yet another embodiment of the invention provides a method for the treatment or prevention of a fungal infection in a substrate, said method comprising administering a compound of formula (1) or a pharmaceutically acceptable salt of such compound having at least one salt forming group or solvate thereof to a substrate in need of such treatment or prevention. In a preferred embodiment provides the substrate is a human being or an animal.

Yet another embodiment comprises a pharmaceutical composition comprising a compound of formula (1) or its solvate or pharmaceutically acceptable salt of such compound having at least one salt forming group for the treatment or prevention of fungal infections Still a further embodiment comprises a process for the preparation of compound of formula (1), or its solvate or pharmaceutically acceptable salt of such compound having at least one salt forming group, said process comprising steps of:

a) Contacting an aldehyde of formula (2)

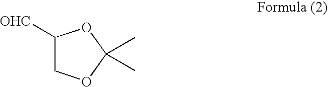

### With a phenyl acetate of formula (3) wherein R1=R2=H, Br, Cl, F and/or methoxy,

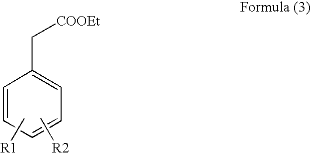

in an organic solvent in presence of a base to obtain the compound of formula (4) wherein R1=R2=H, Br, Cl, F and/or methoxy.

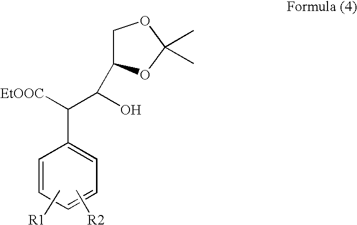

b) Contacting the hydroxy ester of formula (4) with an acidic catalyst in an alcoholic or hydrocarbon solvent to obtain the alcohol of formula (5). The acidic catalyst may be, but is not limited to, p-toluenesulfonic acid (pTSA), HCl, or a combination thereof. The alcoholic solvent may be, but is not limited to, methanol, ethanol, or a combination thereof. The hydrocarbon solvent may be, but is not limited to, toluene.

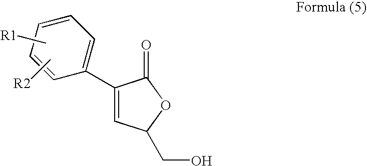

c) Reacting the alcohol of formula (5) with acid chloride in suitable organic solvent in the presence of a catalyst to obtain the compound of formula (1). The organic solvent may be, but is not limited to, chloroform, dichloromethane, or a combination thereof. The catalyst may be, but is not limited to, pyridine, triethylamine, or a combination thereof.

d) converting the compound of formula (1) to its pharmaceutically acceptable salt by adapting conventional methods which are disclosed in “Handbook of Pharmaceutical Salts Properties, Selection and Use” by P H Stahl, C G Wermuth, Wiley-VCH, ISBN: 3906390-26-8).

The compound of formula (1) or its solvates or a pharmaceutically acceptable salt of said compound having at least one salt forming group may be used in the preparation of a pharmaceutically acceptable composition for use in the treatment or prevention of antifungal infections. The pharmaceutical composition is preferably in capsule or tablet form for the treatment or prevention of fungal infection.

A compound of formula (1) may be used along with pharmaceutically acceptable excipients for treatment or prevention of fungal infection in human beings or animals. The excipients may be, but are not limited to, caboxymethylcellulose,lactose,starch, microcrystalline cellulose.

The compounds of present invention may also be used in agrochemical compositions and for prevention and treatment of plant fungal infection.

The compounds of the present invention may be prepared by the route depicted in scheme 1 as shown below:

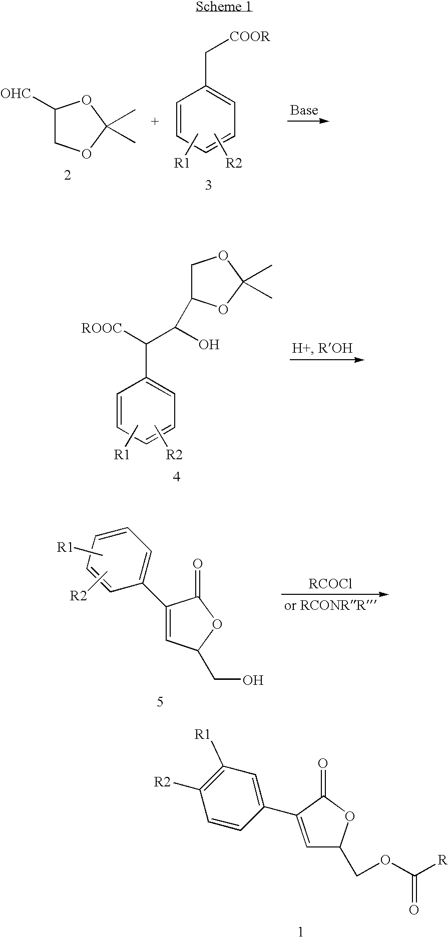

This invention relates to a process for the preparation of 5-aceloxymethyl-3-aryl-2H, 5H-furan-2-ones of the formula (1). More particularly it relates to the process for the preparation of compounds of the formula (1) wherein R₁ and R₂ are each independently hydrogen or halogen or alkoxy; and R=phynyl, allyl, 2-thienyl, 3-thienyl, 4,5-dibromo-2-thienyl, 2-chloroethyl, 3-chloropropyl, 4-chlorobutyl, 3-iodophenyl or 2-pyrrolyl, from 1,2-O-isopropylideneglyceraldehyde of the formula (2).

Accordingly the present invention describes a process for the preparation of 5-aceloxymethyl-3-aryl-2H,5H-furan-2-ones of the formula (1) wherein R₁ and R₂ are each independently of others hydrogen or halogen or alkoxy; and R=phenyl, allyl, 2-thienyl, 3-thienyl, 4,5-dibromo-2-thienyl, 2-chloroethyl, 3-chloropropyl, 4-chlorobutyl, 3-iodophenyl or 2-pyrrolyl. The process comprises reacting 1,2-O-isopropylideneglyceraldehyde of the formula (2) with alkyl 3 and /or 4 substituted phenyl acetate of the formula (3) in a suitable organic solvent in presence of a suitable base and a catalyst at temperature −78 to 10° C. for a suitable period, allowing to come to room temperature, quenching with ammonium chloride solution, extracting with suitable organic solvent, removing the organic solvent, purifying by column chromatography to collect the intermediate of the formula (4), stirring with an acidic catalyst in a suitable solvent at 0-80° C. for a suitable period, purifying by column chromatography to collect the intermediate of the formula (5), reacting with a suitable acid chloride in a suitable solvent in presence of a base at −10 to 30° C. for a suitable period or with a suitable amide in organic solvent at 40- 90° C. for a suitable period, diluting with water, extracting with suitable water immiscible organic solvent, separating the organic layer, washing with water, drying over drying agent, concentrating and purifying the product by column chromatography to collect the required compound of the formula (1),

Structures of some of the preferred alcohols and acid chlorides and/or amides used to obtain preferred compounds of formula (1), by adapting scheme 1 are depicted below:

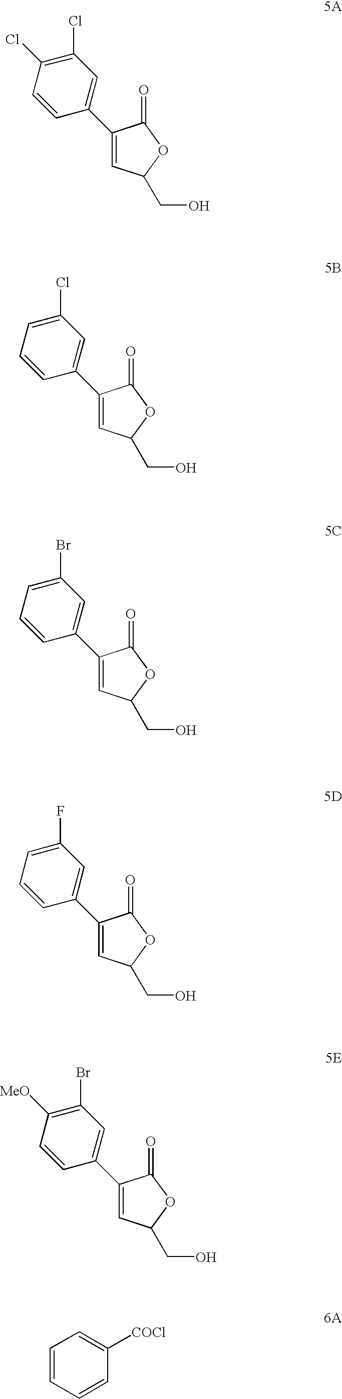

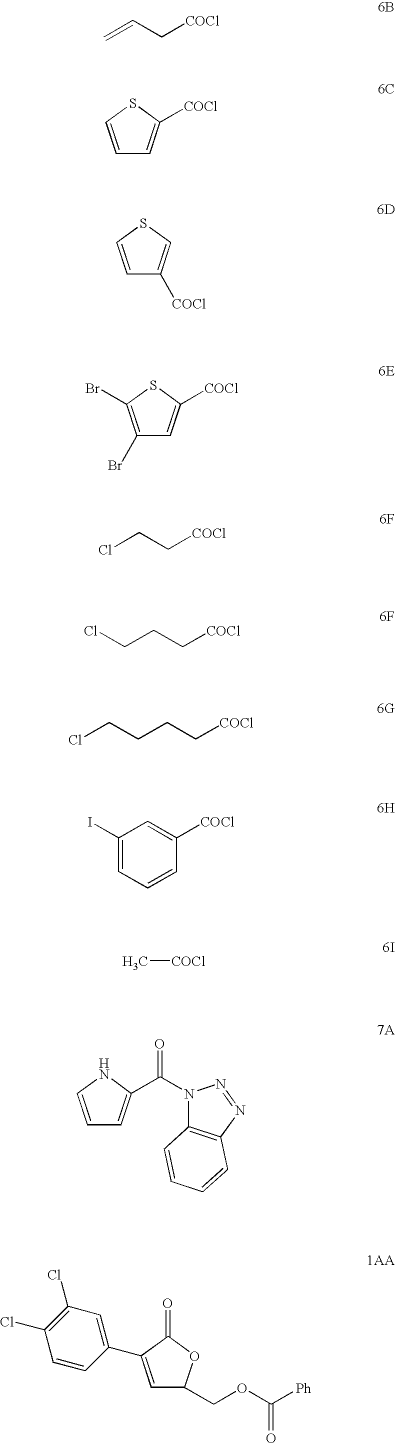

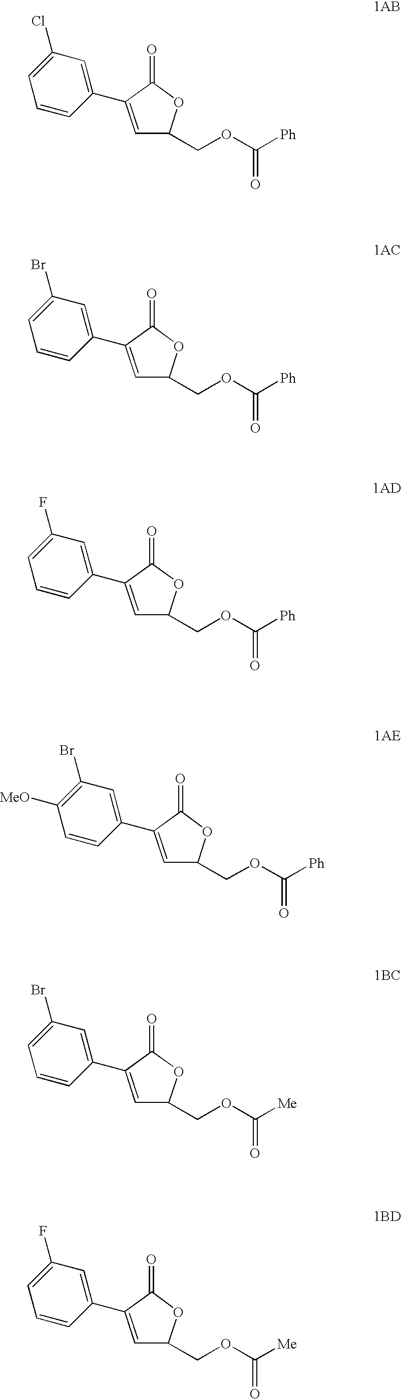

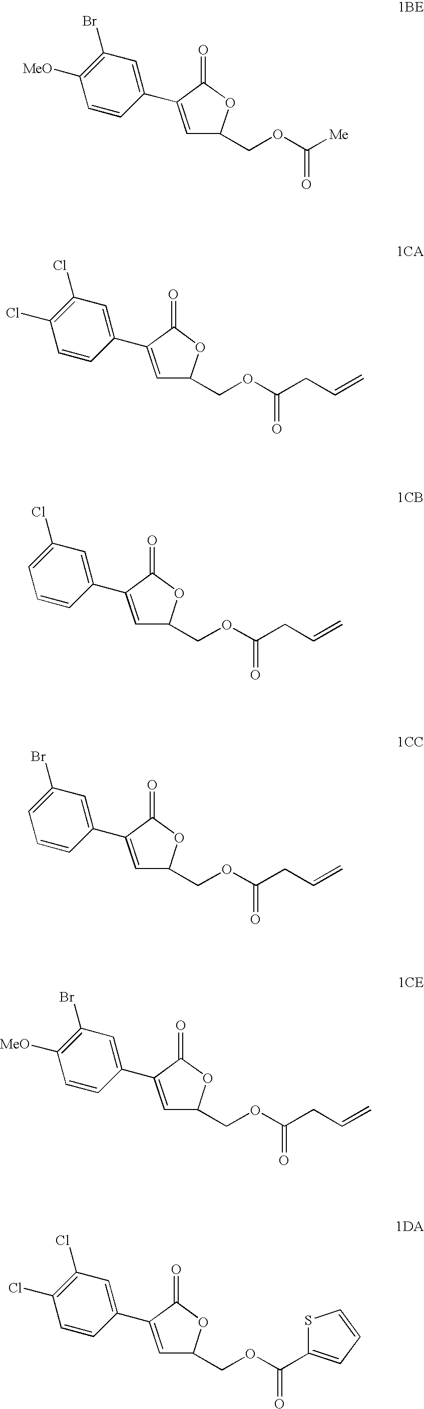

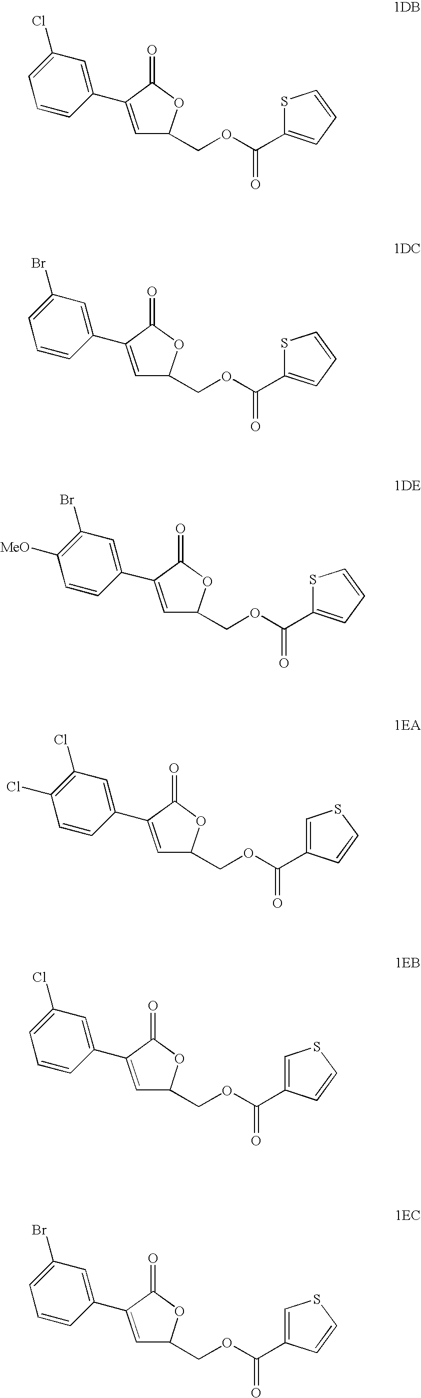

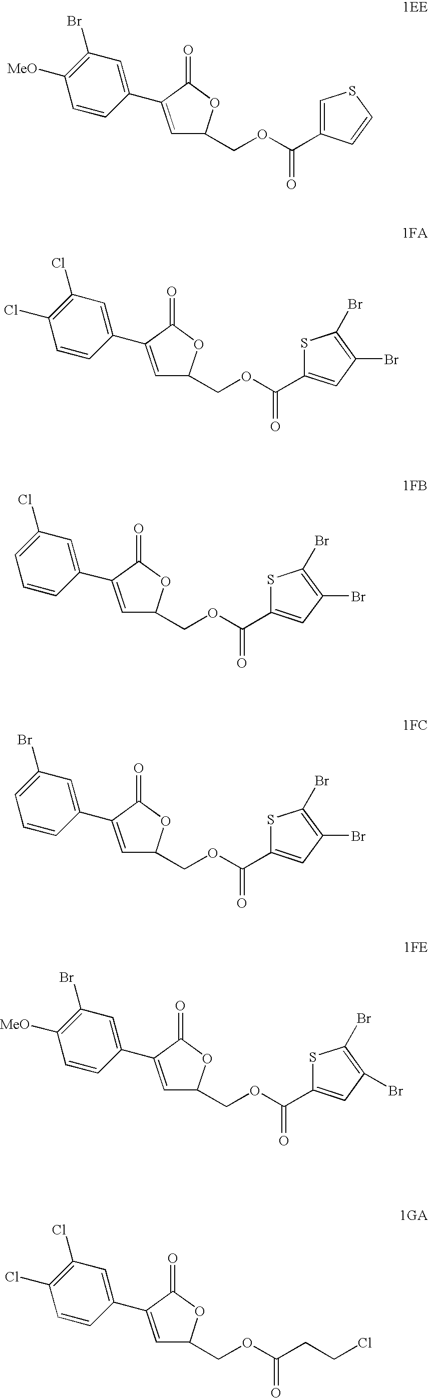

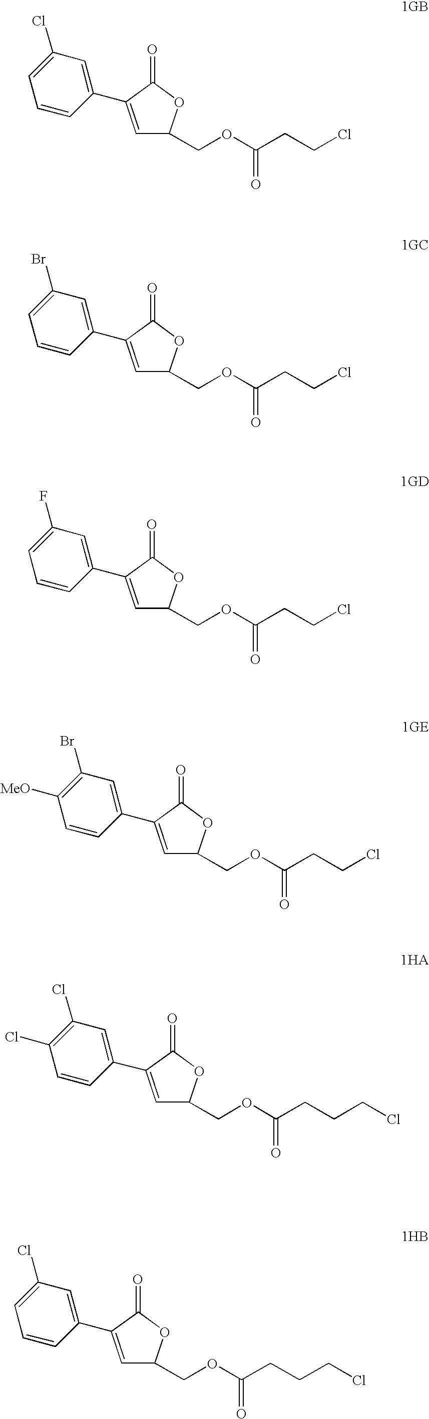

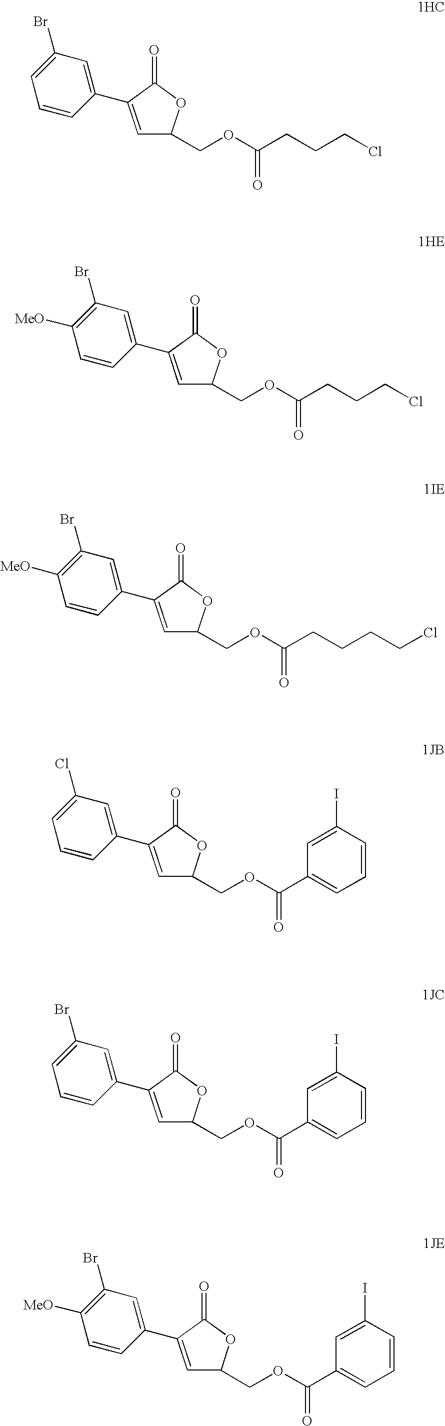

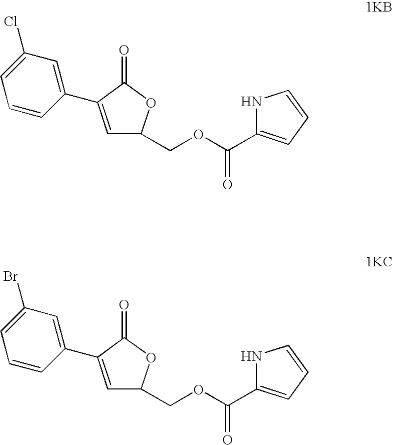

Accordingly, the active compounds of formula (1) of the present invention provide compositions that contain a compound of the present invention together with an excipient and optionally other auxiliary agents. The compositions can be administered in different pharmaceutical preparations, the nature of which will depend, as it is well known, upon the chosen route of administration and the nature of the pathology to be treated. Thus, solid compositions according to the present invention for oral administration include, but are not limited to, dispersible tablets, sustained release (SR) tablets, disintegrating granules pouches, dispersible powders, granules, and capsules.

In tablets, the active component is preferably admixed with at least one inert diluent, such as lactose, starch, mannitol, microcrystalline cellulose, or calcium phosphate; granulating and disintegrating agents, such as corn starch, gelatine, microcrystalline cellulose, or polyvinylpyrrolidone; and lubricating agents, such as magnesium stearate, stearic acid, or talc. The tablets may be uncoated or coated may be SR tablets and can be also formulated by known technique to delay disintegration and absorption in the gastrointestinal tract and, thereby, provide a sustained action over a longer period. Gastric film-coated or enteric film-coated tablets can be made with sugar, gelatin, hydroxypropylcellulose, nor acrylic resins. Tablets with a sustained action may also be obtained using an excipient which provides regressive osmosis, such as the galacturonic acid polymers. Formulations for oral use may also be presented as hard capsules of absorbable material, such as gelatin, wherein the active ingredient is mixed with an inert solid diluent and lubricating agents, or pasty materials, such as ethoxylated saturated glycerides. Soft gelatin capsules are also possible wherein the active ingredient is mixed with water or an oily medium, for example peanut oil, liquid paraffin or olive oil.

Dispersible powders and granules suitable for the preparation of a suspension by the addition of water provide the active ingredient in admixture with a dispersing or wetting agent; a suspending agent, such as sodium carboxymethylcellulose, methylcellulose, hydroxypropylmethylcellulose, sodium alginate, polyvinylpirrolidone, gum tragacanath, xantham gum, or gum acacia; and one or more preservatives, such as methyl or n-propyl p-hydroxybenzoate. Additional excipients, for example sweetening, flavouring and coloring agents may also be present.

Liquid compositions for oral administration include, but are not limited to, emulsions, solutions, suspensions, syrups and elixirs containing commonly used inert diluents, such as distilled water, ethanol, sorbitol, glycerol, or propylene glycol. Aqueous solutions can also be prepared using β-cyclodextrin. Such compositions may also comprise adjuvants, such as wetting agents, suspending agents, sweetening, flavouring, perfuming, preserving agents, or buffers.

Other compositions for oral administration include spray compositions, which may be prepared by known methods, such as those disclosed in S. Published Patent Application No. 2005/0136024 to Stockel, which is incorporated herein by reference. The spray compositions may contain a suitable propellant.

Preparations for injection, according to the present invention, for parenteral administration include, but are not limited to, sterile aqueous or non-aqueous solutions, suspensions or emulsions, in a non-toxic parentally-acceptable diluent or solvent. Examples of aqueous solvents or suspending media are distilled water for injection, Ringer's solution, and isotonic sodium chloride solution. Aqueous solutions can also be prepared using β-cyclodextrin, such as hydroxypropyl-β-cyclodextrin. Examples of non-aqueous solvents or suspending media are propylene glycol, polyethylene glycol, vegetable oils such as olive oil, or alcohols such as ethanol. These compositions may also include adjuvants such as wetting, preserving, emulsifying and dispersing agents. They may be sterilized by one of the known methods or manufactured in the form of sterile solid compositions which can be dissolved in sterile water or some other sterile injectable medium immediately before use. When all of the components are sterile, the injectables will maintain the sterility if they are manufactured in sterile environment.

Preparations for vaginal administration according to the present invention include, but are not limited to, tablets, capsules, softgels, moulded pessaries, creams, foams and vaginal douches. Vaginal tablets preferably provide the active component in admixture with microcrystalline cellulose, pregelatinized starch, lactose, microcrystalline cellulose, pregelatinized starch, polyvidone, and/or magnesium stearate as typical excipients. Soft gelatin capsules (softgels) can be made dispersing the active ingredient in an oily medium, for example liquid paraffin, dimethylpolysiloxane 1000, or hydrogenated soybean oil. Moulded pessaries provide the active ingredient in admixture with a suitable synthetic or semisynehetic base (such as Suppocire® or Novata® types). Low viscosity saturated C₈ to C₁₂ fatty acid glycerides and colloidal silice are also added to improve incorporation and to prevent sedimentation of the active ingredient. Vaginal creams can be prepared as emulsions, with sufficient viscosity to retain their integrity and adhere to the vaginal cavity. Neutral fats, fatty acids, waxes, mineral oils, or fatty acid esters can be used as the oily phase. Water, glycerine, sorbitol solution, or polyethylene glycol are suitable excipients for the aqueous phase. Non-ionic emulsifying agents, such as polyethylene glycol ethers, may also be used. The compositions may also contain preserving, buffering, and/or stiffening agents. Foaming systems can be made using a foamer (dispenser) that is able to transform a solution into a foam. Such systems may include cosolvents, buffers, preservatives, foam stabilizers, or perfumes in an aqueous vehicle. Vaginal douches may contain cosolvents, preservatives, buffers, or perfuming agents in a surfactant rich aqueous solution.

A compound of the invention may also administered in the form of suppositories for rectal administration of the drug, or as creams, ointments, pastes, lotions, gels, sprays, foams, aerosols, solutions, suspensions, or powders for topical use. Such compositions are prepared following conventional procedures well known to those skilled in the art, such as those disclosed in U.S. Published Patent Application No. 2005/0136024 to Stockel, which is incorporated herein by reference.

A compound of the invention may also be administered as a hair or body shampoo. These formulations may be prepared using suitable ionic and/or amphoteric surface-active agents, such as sodium laureth sulfate, triethanolamine laureth sulfate, cocoamidopropyl betaine; thickening agents, such as cocamide DEA, carbomer, sodium chloride, or polyethylene glycol 6000 distearate; and optionally, emollient and superflatting agents, buffers, or preserving and perfuming agents.

The dosage and frequency of dose may vary depending upon the nature and severity of the fungal disease, symptoms, age and body weight of the patient, as well as upon the route of administration. In general, the compounds of the invention will be administered orally or parenterally which can be administered as a single dose or as divided doses.

As used herein, the dosage includes an amount effective for periods of time necessary to achieve the desired result, e.g., sufficient to treat a disease in a subject. An effective amount of the compound of formula (1), as defined herein may vary according to factors such as the disease state, age, and weight of the subject, and the ability of the angiogenesis inhibitor compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of the compound of formula (1) can include a single treatment or, preferably, can include a series of treatments. It will also be appreciated that the effective dosage of the compound of formula (1) used for treatment may increase or decrease over the course of a particular treatment.

The invention is illustrated with the following examples, which should not be construed to limit the scope of the present invention. The features of the present invention will become more apparent from the following description of the inventive concept and the description of the preferred embodiments and appended claims

### EXAMPLE 1

### Preparation of 5-aceloxymethyl-3-aryl-2H, 5H-furan-2-ones of the formula (1) by reaction of alcohols 5 with acid chlorides 6

To a solution of alcohols 5 (1 mmole) and pyridine (1 mmole) in 10 mL of DCM was added acid chloride 6 (1.1 mmole) 0° C. The reaction mixture was stirred at rt for 2 h and then washed with water (2×10 mL), dil. HCl (2×10 mL), water (2×10 mL), dried over anhydr. Na₂SO₄ and concentrated. The residue was purified by column chromatography to yield the compounds of formula 1.

The following compounds were prepared by the procedure given above:

### 1) 3,4-dichlorophenyl-5-benzoyloxymethyl-2H,5H-furan-2-one (1AA)

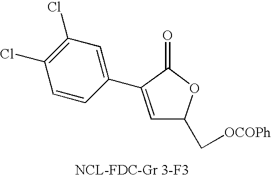

¹H NMR (CDCl₃): δ 4.66 (d, J=5 Hz, 2H), 5.38-5.45 (m, 1H), 7.39-7.50 (m, 3H), 7.55-7.59 (m, 1H), 7.63 (bs, 1H), 7.70 (dd, J=10, 2 Hz, 1H), 7.96-8.01 (m, 3H).

### 2) Preparation of 3-chlorophenyl-5-benzoyloxymethyl-2H,5H-furan-2-one (1AB)

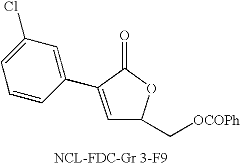

¹H NMR (CDCl₃): δ 4.55-4.80 (m, 2H), 5.35-5.47 (m, 1H), 7.32-7.49 (m, 4H), 7.52-7.67 (m, 2H), 7.71-7.80 (m, 1H), 7.85 (bs, 1H), 7.93-8.20 (m, 2H).

### 3) Preparation of 3-bromophenyl-5-benzoyloxymethyl-2H,5H-furan-2-one (1AC)

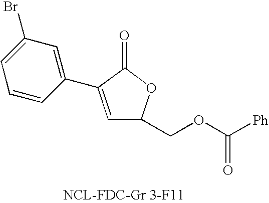

¹H NMR (CDCl₃): δ 4.52-4.83 (m, 2H), 5.30-5.55 (m, 1H), 7.22-7.70 (m, 6H), 7.80 (bd, J=8 Hz, 1H), 7.95-8.20 (m, 3H).

### 4) Preparation of 3-fluorophenyl-5-benzoyloxymethyl-2H,5H-furan-2-one (1AD)

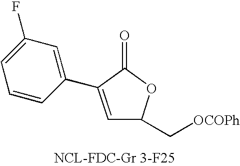

¹H NMR (CDCl₃): δ 4.55-4.76 (m, 2H), 5.30-5.45 (m, 1H), 7.00-7.14 (m, 1H), 7.25-7.48 (m, 4H), 7.51-7.70 (m, 4H), 7.90-8.10 (m, 1H).

### 5) Preparation of 3-bromo-4-methoxyphenyl-5-benzoyloxymethyl-2H,5H-furan-2-one (1AE)

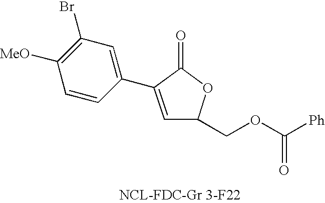

¹H NMR (CDCl₃): δ 3.96 (s, 3H), 4.57-4.75 (m, 2H), 5.37-5.45 (m, 1H), 6.95 (d, J=10 Hz, 1H), 7.40-7.63 (m, 4H), 7.91 (dd, J=8, 2 Hz, 1H), 8.00-8.09 (m, 3H).

### 6) Preparation of 3-bromophenyl-5-acetxymethyl-2H,5H-furan-2-one (1BC)

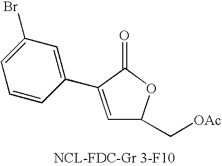

¹H NMR (CDCl₃): δ 2.08 (s, 3H), 4.31-4.45 (m, 2H), 5.21-5.30 (m, 1H), 7.33 (d, J=8 Hz, 1H), 7.50-7.61 (m, 2H), 7.82 (bd, J=8 Hz, 1H), 8.01 (bs, 1H).

### 7) Preparation of 3-fluorophenyl-5-acetxymethyl-2H,5H-furan-2-one (1BD)

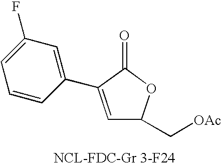

¹H NMR (CDCl₃): 6 2.05 (s, 3H), 4.30-4.46 (m, 2H), 5.20-5.29 (m, 1H), 7.00-7.16 (m, 1H), 7.30-7.45 (m, 1H), 7.51-7.70 (m, 3H).

### 8) Preparation of 3-bromo-4-methoxyphenyl-5-acetoxymethyl-2H,5H-furan-2-one (1BE)

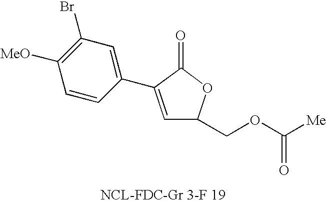

¹H NMR (CDCl₃): δ 2.07 (s, 3H), 3.93 (s, 3H), 4.25-4.48 (m, 2H), 5.18-5.30 (m, 1H), 6.93 (d, J=8 Hz, 1H), 7.42 (bs, 1H), 7.90 (bdd, J=8 Hz, 1H), 8.01 (bs, 1H).

### 9) Preparation of 3,4-dichlorophenyl-5-allyloyloxymethyl-2H,5H-furan-2-one (1CA)

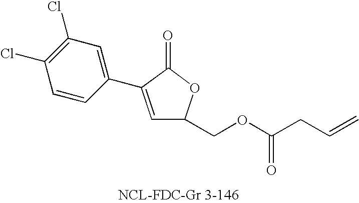

¹H NMR (CDCl₃): δ 3.07 (d, J=8 Hz, 2H), 4.45 (d, J=4 Hz, 2H), 5.02-5.45 (m, 3H), 5.70-5.95 (m, 1H), 7.45 (d, J=8 Hz, 1H), 7.53 (d, J=2 Hz, 1H), 7.70 (dd, J=8, 2 Hz, 1H), 7.96 (d, J=2 Hz, 1H).

### 10) Preparation of 3-chlorophenyl-5-allyloyloxymethyl-2H,5H-furan-2-one (1CB)

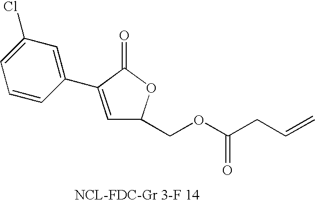

¹H NMR (CDCl₃): δ 3.10 (d, J=6 Hz, 2H), 4.45 (d, J=4 Hz, 2H), 5.02-5.35 (m, 3H), 5.75-5.95 (m, 1H), 7.35-7.45 (m, 2H), 7.54 (d, J=2 Hz, 1H), 7.70-7.80 (m, 1H), 7.85 (bs, 1H).

### 11) Preparation of 3-bromophenyl-5-allyloyloxymethyl-2H,5H-furan-2-one (1CC)

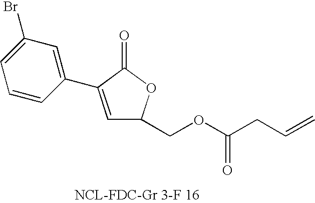

¹H NMR (CDCl₃): δ 3.11 (d, J=8 Hz, 2H), 4.40-4.50 (m, 2H), 5.05-5.30 (m, 3H), 5.70-5.95 (m, 1H), 7.25-7.40 (m, 2H), 7.54-7.60 (m, 1H), 7.81 (d, J=8 Hz, 1H), 7.99 (d, J=2 Hz, 1H).

### 12) Preparation of 3-bromo-4-methoxyphenyl-5-allyloyloxymethyl-2H,5H-furan-2-one (ICE)

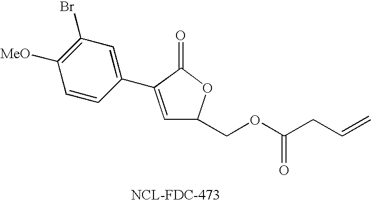

¹H NMR (CDCl₃): δ 3.08 (d, J=8 Hz, 2H), 3.94 (s, 3H), 4.40 (d, J=6 Hz, 2H), 5.05-5.30 (m, 3H), 5.70-5.95 (m, 1H), 6.92 (d, J=10 Hz, 1H), 7.40 (d, J=2 Hz, 1H), 7.85 (dd, J=10, 2 Hz, 1H), 8.02 (d, J=2 Hz, 1H).

### 13) Preparation of 4-(3,4-dichlorophenyl)-5-oxo-2,5-dihydrofuran-2-ylmethyl thiophene-2-carboxylate (1DA)

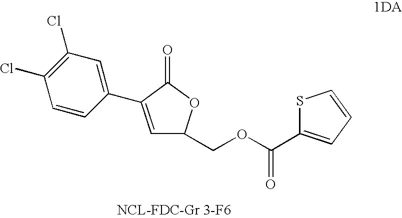

¹H NMR (CDCl₃): δ 4.60 (d, J=5 Hz, 2H), 5.28-5.50 (m, 1H), 7.05-7.20 (m, 1H) 7.40-7.85 (m, 5H), 7.93 (bs, 1H).

### 14) Preparation of 4-(3-chlorophenyl)-5-oxo-2,5-dihydrofuran-2-ylmethyl thiophene-2-carboxylate (1DB)

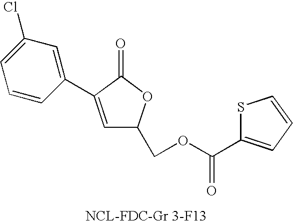

¹H NMR (CDCl₃): δ 4.64 (d, J=4 Hz, 2H), 5.33-5.42 (m, 1H), 7.07-7.18 (m, 1H), 7.33-7.45 (m, 2H), 7.55-7.61 (m, 2H), 7.70-7.95 (m, 3H).

### 15) Preparation of 4-(3-bromophenyl)-5-oxo-2,5-dihydrofuran-2-ylmethyl thiophene-2-carboxylate (1DC)

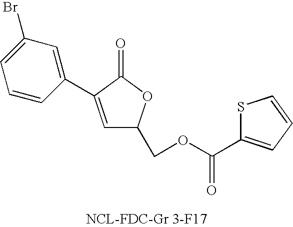

¹H NMR (CDCl₃): δ 4.54-4.65 (m, 2H), 5.31-5.40 (m, 1H), 7.05-7.15 (m, 1H), 7.20-7.35 (m, 1H), 7.45-7.68 (m, 3H), 7.72-7.83 (m, 2H), 7.97 (bs, 1H).

### 16) Preparation of 4-(3-bromo-4-methoxyphenyl)-5-oxo-2,5-dihydrofuran-2-ylmethyl thiophene-2-carboxylate (1DE)

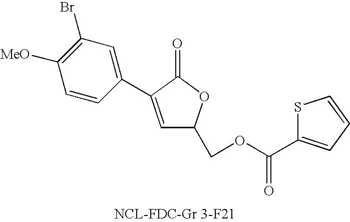

¹H NMR (CDCl₃): δ 3.93 (s, 3H), 4.51-4.70 (m, 2H), 5.31-5.41 (m, 1H), 6.93 (d, J=8 Hz, 1H), 7.05-7.13 (m, 1 H), 7.48 (d, J=2 Hz, 1H), 7.59 (bd, J=6 Hz, 1H), 7.74-7.93 (m, 2H), 8.01 (d, J=2 Hz, 1H).

### 17) Preparation of 4-(3,4-dichlorophenyl)-5-oxo-2,5-dihydrofuran-2-ylmethyl thiophene-3-carboxylate (1EA)

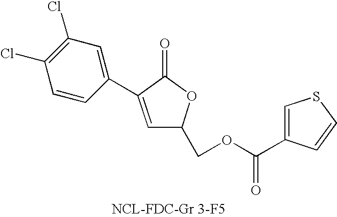

¹H NMR (CDCl₃): δ 4.63 (d, J=5 Hz, 2H), 5.35-5.45 (m, 1H), 7.25-7.35 (m, 1H), 7.40-7.53 (m, 2H), 7.60-7.75 (m, 2H), 7.95 (d, J=2 Hz, 1H), 8.10 (d, J=2 Hz, 1H).

### 18) Preparation of 4-(3-chlorophenyl)-5-oxo-2,5-dihydrofuran-2-ylmethyl thiophene-3-carboxylate (1EB)

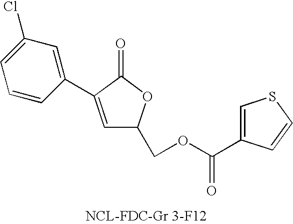

¹H NMR (CDCl₃): δ 4.55-4.70 (m, 2H), 5.33-5.45 (m, 1H), 7.22-7.43 (m, 3H), 7.49 (d, J=8 Hz, 1H), 7.63 (bs, 1H), 7.75 (bd, J=8 Hz, 1H), 7.85 (bs, 1H), 8.12 (d, J=2 Hz, 1H).

### 19) Preparation of 4-(3-bromophenyl)-5-oxo-2,5-dihydrofuran-2-ylmethyl thiophene-3-carboxylate (1EC)

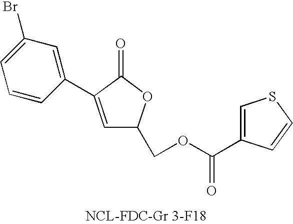

¹H NMR (CDCl₃): δ 4.52-4.65 (m, 2H), 5.35-5.43 (m, 1H), 7.25-7.35 (m, 2H), 7.45-7.58 (m, 2 H), 7.62 (d, J=2 Hz, 1H), 7.79 (d, J=8 Hz, 1H), 7.98 (bs, 1H), 8.10 (bs, 1H).

### 20) Preparation of 4-(3-bromo-4-methoxyphenyl)-5-oxo-2,5-dihydrofuran-2-ylmethyl thiophene-3-carboxylate (1EE)

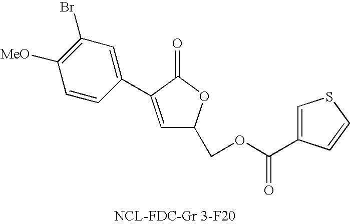

¹H NMR (CDCl₃): δ 3.85 (s, 3H), 4.40-4.62 (m, 2H), 5.23-5.33 (m, 1H), 6.84 (d, J=8 Hz, 1H), 7.18-7.28 (m, 1 H), 737-7.44 (m, 2H), 7.79 (dd, J=8, 2 Hz, 1H), 7.92 (bs, 1H), 8.03 (bs, 1H).

### 21) Preparation of 4-(3,4-dichlorophenyl)-5-oxo-2,5-dihydrofuran-2-ylmethyl 4,5-dibromothiophene-2-carboxylate (1FA)

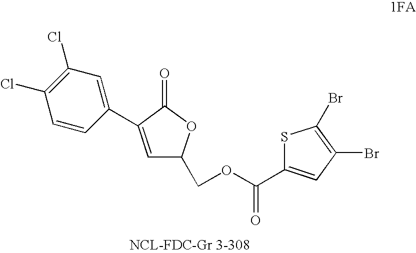

¹H NMR (CDCl₃): δ 4.52-4.72 (m, 2H), 5.32-5.41 (m, 1H), 7.50 (d, J=8 Hz, 1H), 7.59 (bs, 2H), 7.72 (dd, J=8, 2 Hz, 1H), 7.97 (d, J=2 Hz, 1H).

### 22) Preparation of 4-(3-chlorophenyl)-5-oxo-2,5-dihydrofuran-2-ylmethyl 4,5-dibromothiophene-2-carboxylate (1FB)

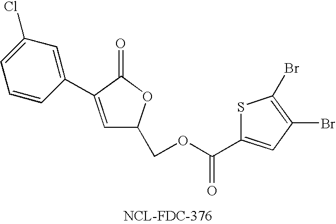

¹H NMR (CDCl₃): δ 4.50-4.71 (m, 2H), 5.30-5.40 (m, 1H), 7.29-7.43 (m, 2H), 7.57 (bs, 2H), 7.68-7.77 (m, 1H), 7.84 (bs, 1H).

### 23) Preparation of 4-(3-bromophenyl)-5-oxo-2,5-dihydrofuran-2-ylmethyl 4,5-dibromothiophene-2-carboxylate (1FC)

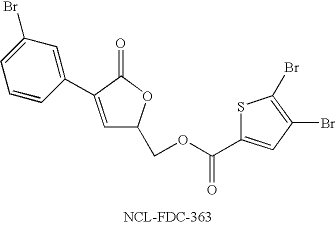

¹H NMR (CDCl₃): δ 4.45-4.72 (m, 2H), 5.28-5.40 (m, 1H), 7.30 (t, J=8 Hz, 1H), 7.44-7.63 (m, 3H), 7.80 (d, J=8 Hz, 1H), 7.98 (s, 1H).

### 24) Preparation of 4-(3-bromo-4-methoxyphenyl)-5-oxo-2,5-dihydrofuran-2-ylmethyl 4,5-dibromothiophene-2-carboxylate (1FE)

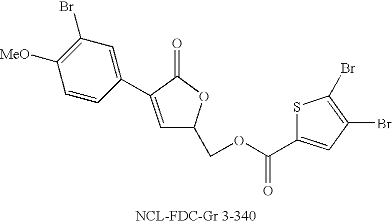

¹H NMR (CDCl₃): δ 3.93 (s, 3H), 4.46-4.71 (m, 2H), 5.29-5.40 (m, 1H), 6.93 (d, J=2Hz, Hz, 1H), 7.44 (d, J=2 Hz, 1H), 7.58 (s, 1H), 7.88 (dd, J=8, 2 Hz, 1H), 8.00 (d, J=2 Hz, 1H).

### 25) Preparation of 4-(3,4-dichlorophenyl)-5-oxo-2,5-dihydrofuran-2-ylmethyl 3-chloropropionate (1GA)

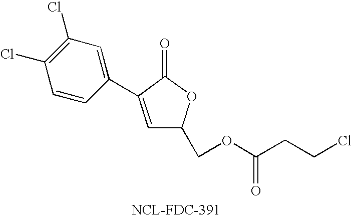

¹H NMR (CDCl₃): δ 2.79 (t, J=7 Hz, 2H), 3.72 (t, J=7 Hz, 2H), 4.49 (d, J=3 Hz, 2H), 5.23-5.34 (m, 1H), 7.48 (d, J=8 Hz, 1H), 7.57 (s, 1 H), 7.71 (d, J=8 Hz, 1H), 7.98 (bs, 1H).

### 26) Preparation of 4-(3-chlorophenyl)-5-oxo-2,5-dihydrofuran-2-ylmethyl 3-chloropropionate (1GB)

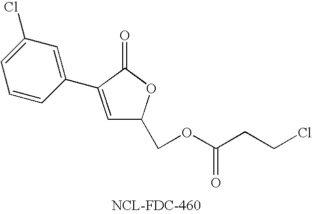

¹H NMR (CDCl₃): δ 2.80 (t, J=6 Hz, 2H), 3.71 (t, J=6 Hz, 2H), 4.47 (d, J=3 Hz, 2H), 5.25-5.36 (m, 1H), 7.30-7.42 (m, 2H), 7.57 (bs, 1 H), 7.74 (bd, J=6 Hz, 1H), 7.85 (bs, 1H).

### 27) Preparation of 4-(3-bromophenyl)-5-oxo-2,5-dihydrofuran-2-ylmethyl 3-chloropropionate (1GC)

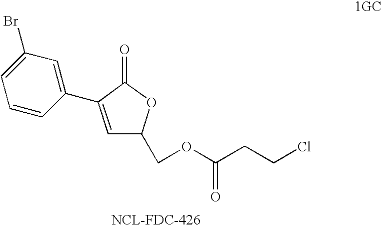

¹H NMR (CDCl₃): δ 2.81 (t, J=6 Hz, 2H), 3.72 (t, J=6 Hz, 2H), 4.48 (d, J=4 Hz, 2H), 5.25-5.35 (m, 1H), 7.30 (t, J=9 Hz, 1H), 7.47-7.60 (m, 2 H), 7.81 (d, J=9 Hz, 1H), 8.00 (bs, 1H).

### 28) Preparation of 4-(3-bromo-4-methoxyphenyl)-5-oxo-2,5-dihydrofuran-2-ylmethyl 3-chloropropionate (1GE)

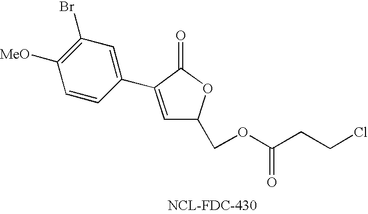

¹H NMR (CDCl₃): δ 2.85 (t, J=8 Hz, 2H), 3.76 (t, J=8 Hz, 2H), 3.97 (s, 3H), 4.49 (d, J=6 Hz, 2H), 5.25-5.36 (m, 1H), 6.96 (d, J=10 Hz, 1H), 7.47 (d, J=2 Hz, 1 H), 7.92 (dd, J=10, 2 Hz, 1H), 8.06 (d J=2 Hz, 1H).

### 29) Preparation of 4-(3,4-dichlorophenyl)-5-oxo-2,5-dihydrofuran-2-ylmethyl 4-chlorobutyrate (1HA)

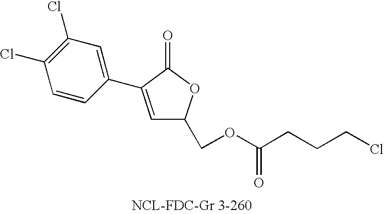

¹H NMR (CDCl₃): δ 2.01-2.13 (m, 2H), 2.53 (t, J=6 Hz, 2H), 3.56 (t, J=6 Hz, 2H), 4.42 (d, J=4 Hz, 2H), 5.22-5.32 (m, 1H), 7.50 (d, J=10 Hz, 1H), 7.56 (d, J=1 Hz, 1H), 7.74 (dd, J=10, 1 Hz, 1H), 7.99 (d, J=2 Hz, 1H).

### 30) Preparation of 4-(3-chlorophenyl)-5-oxo-2,5-dihydrofuran-2-ylmethyl 4-chlorobutyrate (1HB)

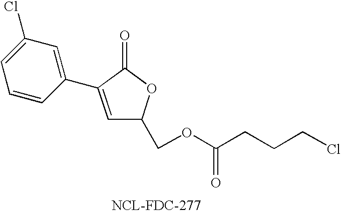

¹H NMR (CDCl₃): δ 2.05 (quintet, J=8 Hz, 2H), 2.50 (t, J=8 Hz, 2H), 3.04 (t, J=8 Hz, 2H), 4.32-4.51 (m, 2H), 5.20-5.31 (m, 1H), 7.29-7.45 (m, 2H), 7.55 (s, 1 H), 7.65-7.80 (m, 1H), 7.85 (s, 1H).

### 31) Preparation of 4-(3-bromophenyl)-5-oxo-2,5-dihydrofuran-2-ylmethyl 4-chlorobutyrate (1HC)

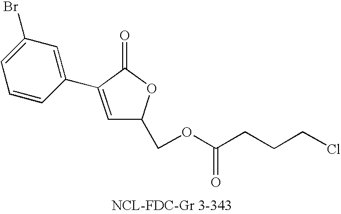

¹H NMR (CDCl₃): δ 2.05 (quintet, J=6 Hz, 2H), 2.52 (t, J=6 Hz, 2H), 3.56 (t, J=6Hz, 2H), 4.41 (d, J=2 Hz, 2H), 5.20-5.32 (m, 1H), 7.31 (t, J=8 Hz, 1H), 7.45-5.63 (m, 2H), 7.82 (d, J=8 Hz, 1H), 7.98 (s, 1H).

32) Preparation of 4-(3-fluorophenyl)-5-oxo-2,5-dihydrofuran-2-ylmethyl 4-chlorobutyrate (1HD)

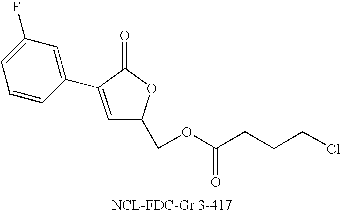

¹H NMR (CDCl₃): δ 2.06 (quintet, J=6 Hz, 2H), 2.53 (t, J=6 Hz, 2H), 3.56 (t, J=6 Hz, 2H), 4.32-4.56 (m, 2H), 5.20-5.45 (m, 1H), 7.05-7.25 (m, 1H), 7.53-7.51 (m, 3H).

### 33) Preparation of 4-(3-bromo-4-methoxyphenyl)-5-oxo-2,5-dihydrofuran-2-ylmethyl 4-chlorobutyrate (1HE)

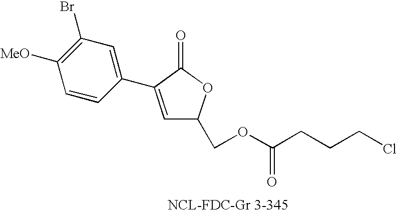

¹H NMR (CDCl₃): δ 2.05 (quintet, J=6 Hz, 2H), 2.52 (t, J=6 Hz, 2H), 3.55 (t, J=6 Hz, 2H), 3.93 (s, 3H), 4.30-4.50 (m, 2H), 5.16-5.28 (m, 1H), 6.92 (d, J=8 Hz, 1H), 7.41 (bs, 1H), 7.89 (d, J=8 Hz, 1H), 8.00 (bs, 1H).

### 34) Preparation of 4-(3-bromo-4-methoxyphenyl)-5-oxo-2,5-dihydrofuran-2-ylmethyl 5-chlorovalerate (1IE)

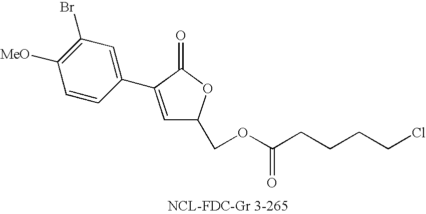

¹H NMR (CDCl₃): δ 1.65-1.80 (m, 4H), 2.30-2.45 (m, 2H), 3.43-3.55 (m, 2H), 3.94 (s, 3H), 4.37-4.45 (m, 2H), 5.20-5.30 (m, 1H), 6.94 (d, J=8 Hz, 1H), 7.42 (bs, 1H), 7.89 (d, J=8 Hz, 1H), 8.02 (bs, 1H).

### 35) Preparation of 4-(3-chlorophenyl)-5-oxo-2,5-dihydrofuran-2-ylmethyl 3-iodobenzoate (1JB)

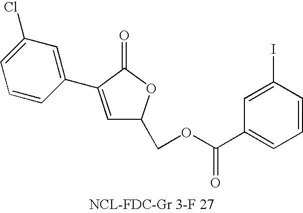

¹H NMR (CDCl₃): δ 4.57 (dd, J=12, 4 Hz, 1H), 4.68 (dd, J=12, 4 Hz, 1H), 5.34-5.44 (m, 1H), 7.17 (t, J=8 Hz, 1H), 7.31-7.42 (m, 2H), 7.60 (s, 1H), 7.70-7.79 (m, 1H), 7.82 (s, 1H), 7.85-8.00 (m, 2H), 8.31 (bs, 1H).

### 36) Preparation of 4-(3-bromophenyl)-5-oxo-2,5-dihydrofuran-2-ylmethyl 3-iodobenzoate (1JC)

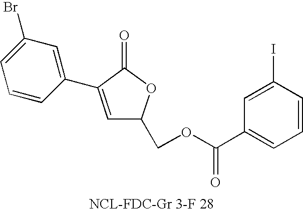

¹H NMR (CDCl₃): δ 4.55-4.85 (m, 2H), 5.38 -5.55 (m, 1H), 7.23 (t, J=8 Hz, 1H), 7.35 (t, J=8 Hz, 1H), 7.52-7.75 (m, 2H), 7.80-8.18 (m, 4H), 8.37 (bs, 1H).

### 37) Preparation of 4-(3-bromo-4-methoxyphenyl)-5-oxo-2,5-dihydrofuran-2-ylmethyl 3-iodobenzoate (1JE)

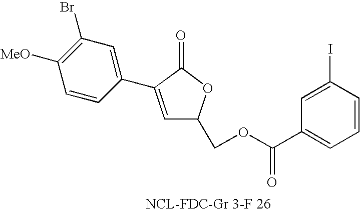

¹H NMR (CDCl₃): δ 3.93 (s, 3H), 4.48-4.71 (m, 2H), 5.29-5.40 (m, 1H), 6.90 (d, J=8 Hz, 1H), 7.17 (t, J=8 Hz, 1H), 7.47 (bs, 1H), 7.84-8.02 (m, 4H), 8.32 (bs, 1H).

### EXAMPLE 2

### Preparation of 5-aceloxymethyl-3-aryl-2H, 5H-furan-2-ones of the formula (1) by reaction of alcohols 5 with amide 7

To a solution of benzotriazole (148 mg, 1.25 mmole), pyrrole-2-carboxylic acid (138 mg, 1.25 mmole) in 5 mL of dry THF was added 0.091 mL (1.25 mmole) of SOCl₂ at −10-0° C. The resulting mixture was stirred for 15 minutes followed by the addition of the alcohol 5 (0.81 mmole) in 2 mL of THF. The resultant mixture was refluxed under argon atmosphere for 10-20 h. The reaction mixture was diluted with 5 mL of ethyl acetate and washed with sat. Na₂SO₄ solution (2×5 mL), water (2×5 mL) and concentrated. The residue was purified by column chromatography to afford the 5-aceloxymethyl-3-aryl-2H,5H-furan-2-ones of the formula (1) in 20-30% yield. The following compounds were prepared by the procedure given above:

### 1) 4-(3-chlorophenyl)-5-oxo-2,5-dihydrofuran-2-ylmethyl pyrrole-2-carboxylate (1KB)

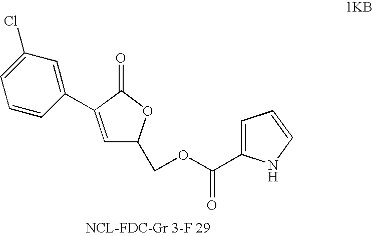

¹H NMR (CDCl₃): δ 4.52 (d, J=4 Hz, 2H), 5.23-5.34 (m, 1H), 6.12-6.24 (m, 1H), 6.80-6.96 (m, 2H), 7.21-7.37 (m, 2H), 7.53 (bs, 1H), 7.65 (d, J=6 Hz, 1H), 7.75 (s, 1H), 9.13 (bs, 1H).

### 2) 4-(3-bromophenyl)-5-oxo-2,5-dihydrofuran-2-ylmethyl pyrrole-2-carboxylate (1KC)

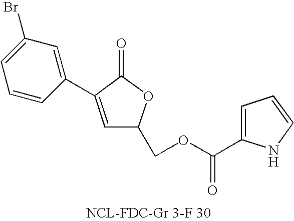

¹H NMR (CDCl₃): δ 4.53 (d, J=4 Hz, 2H), 5.25-5.35 (m, 1H), 6.18-6.24 (m, 1H), 6.85-6.95 (m, 2H), 7.23 (t, J=8 Hz, 1H), 7.47 (d, J=8 Hz, 1H), 7.54 (d, J=2 Hz, 1H), 7.73 (d, J=8 Hz, 1H), 7.91 (bs, 1H), 9.14 (bs, 1H).

### EXAMPLE 3

### Preparation of 3-aryl-5-hydroxymethyl-2H, 5H-furan-2-ones of formula (5)

Step 1: Preparation of hydroxy ester of formula (4): To a solution of 32.5 mmoles of LDA (prepared by the addition of 20.4 mL of 1.6 molar nBuLi to 4.98 mL of diisopropylamine in 90 mL of THF at 0° C.) was added dropwise a solution of phenyl acetates of formula (3) wherein R1=R2=H, Br, Cl, F and/or methoxy (35.8 mmole) in 20 mL of THF at −78° C. The resulting mixture was stirred at that temperature for 30 minutes followed by the addition of 8 mL of HMPA and stirred for further 20 minutes. To this mixture was added dropwise a solution of aldehyde of formula (2) (5.8 g, 32.5 mmole) in 20 mL of THF and the stirring was continued at −78° C. for 1 h. The reaction mixture was allowed to warm to room temperature followed by quenching with ammonium chloride solution. The reaction mixture was diluted with 100 mL of ethyl acetate, washed with water (2×100 mL), 1 N HCl solution (2×50 mL), water (2×100 mL), dried over Na₂SO₄ and concentrated. The residue was purified by column chromatography to afford the hydroxy ester of formula (4) as a pale yellow pasty mass. Yield 50-60%.

Step 2: Preparation of the alcohol 5

A solution of the hydroxy ester of formula (4) (6.96 mmole) and cat. pTSA (50 mg) in 20 mL of distilled MeOH was stirred at 40° C. for 3 days. The reaction mixture was concentrated and the residue was purified by flash column chromatography to give the alcohol of the formula (5) as a white solid. Yield 30-50%.

The following compounds were prepared by the procedure described above for the alcohol of the formula (5):

### 1) 3-(3-chlorophenyl)-5-hydroxymethyl-2H,5H-furan-2-one of the formula (5B)

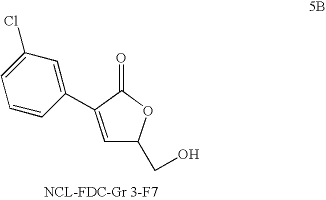

¹H NMR (CDCl₃): δ 3.84 (dd, J=14, 6 Hz, 1H), 4.04 (dd, J=14, 6 Hz, 1H), 5.10-5.25 (m, 1H), 7.28-7.45 (m, 2H), 7.61 (d, J=2 Hz, 1H), 7.71-7.80 (m, 1H), 7.85 (bs, 1H).

### 2) 3-(3-bromophenyl)-5-hydroxymethyl-2H,5H-furan-2-one of the formula (5c)

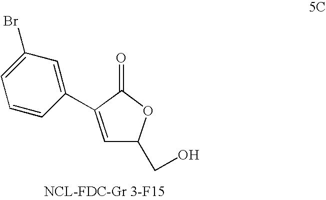

¹H NMR (CDCl₃): δ 3.78-4.10 (m, 2H), 5.10-5.20 (m, 1H), 7.27 (t, J=8 Hz, 1), 7.51 (d, J=8 Hz, 1H), 7.62 (s, 1H), 7.78 (d, J=8 Hz, 1H), 7.98 (s, 1H).

### 3) 3-(3-bromo-4-methoxyphenyl)-5-hydroxymethyl-2H,5H-furan-2-one of the formula (5E)

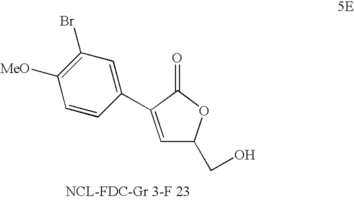

¹H NMR (CDCl₃+DMSO-d₆): δ 3.78-3.93 (m, 2H), 3.95 (s, 3H), 5.07-5.17 (m, 1H), 6.95 (d, J=10 Hz, 1H), 7.64 (d, J=3 Hz, 1H), 7.64 (d, J=3 Hz, 1H), 7.87 (dd, J=10, 3 Hz, 1H), 8.08 (d, J=2 Hz, 1H).

### EXAMPLE 4

### Antifungal Activity Testing

The compound of formula (1) and their pharmaceutically acceptable salts are antifungal agents effective to a greater or lesser extent, and useful in treating fungal infectionsin animals and humans, especially those caused by Calibicans, Aspergillus and Fusarium.

In vitro evaluation of antifungal activity can be performed by determining the minimum inhibitory concentration.

Anti-fungal susceptibility testing of these anti-fungal compounds was done by conventional method using soyabean casein digest broth. Known anti-fungal agents like Fluconazole and amphotericin-B were used as positive control. End points were determined after 48 hours visually and by using Spectrophotometer wherever necessary. Different dilutions were tried and the set of experiments.

Antifungal activity of these compounds also extends to Aspergillus and Fusarium. The activity seen in compound of formula (1) as against these strains suggests that it exhibits broad spectrum antifungal activity.

The results are enumerated in Table 1 below:

TABLE 1

| | | | Activity against organisms | | |
|---|---|---|---|---|---|
| Sr | | | In μg/ml | | |
| no | Code no | Structure | C. albicans | A. niger | F. proliferatum |
| 01 | 1AA | R1 = R2 = Cl, R = Ph | 4 | 2 | 1 |
| 02 | 1AB | R1 = Cl, R2 = H, | 2 | 2 | 0.5 |
| | | R = Ph | | | |
| 03 | 1AC | R1 = Br, R2 = H, | 2 | 4 | 0.5 |
| | | R = Ph | | | |
| 04 | 1AD | R1 = F, R2 = H, | 1–2 | 1–2 | 1–2 |
| | | R = Ph | | | |
| 05 | 1AE | R1 = Br, R2 = OMe, | NI till 2 | NI till 2 | NI till 2 |
| | | R = Ph | | | |
| 06 | 1BC | R1 = Br, R2 = H, | 2 | 4 | 2 |
| | | R = Me | | | |
| 07 | 1BD | R1 = F, R2 = H, | 1–2 | 1–2 | 1–2 |
| | | R = Me | | | |
| 08 | 1BE | R1 = Br, R2 = OMe, | 2–4 | 4–8 | 8–16 |
| | | R = Me | | | |
| 09 | 1CA | R1 = R2 = Cl, R = allyl | 2 | 1 | 1 |
| 10 | 1CB | R1 = Cl, R2 = H, | 4 | 8 | 2 |
| | | R = -allyl | | | |
| 11 | 1CC | R1 = Br, R2 = H, | 4 | 8 | 2 |
| | | R = CO-allyl | | | |
| 12 | 1CE | R1 = Br, R2 = OMe, | 2–4 | NI till 8 | NI till 8 |
| | | R = -allyl | | | |
| 13 | 1DA | R1 = R2 = Cl, R = 2- | 2 | 1 | 2 |
| | | thienyl | | | |
| 14 | 1DB | R1 = Cl, R2 = H, | 2 | 4 | 1 |
| | | R = 2-thienyl | | | |
| 15 | 1DC | R1 = Br, R2 = H, | 2 | 8 | 2 |
| | | R = 2-thienyl | | | |
| 16 | 1DE | R1 = Br, R2 = OMe, | NI till 2 | NI till 2 | NI till 2 |
| | | R = 2- | | | |
| | | thienyl | | | |
| 17 | 1EA | R1 = R2 = Cl, R = 3- | 2 | 1 | 2 |
| | | thienyl | | | |
| 18 | 1EB | R1 = Cl, R2 = H, | 4 | 4 | 2 |
| | | R = 3-thienyl | | | |
| 19 | 1EC | R1 = Br, R2 = H, | 2 | 2 | 1 |
| | | R = 3-thienyl | | | |
| 20 | 1EE | R1 = Br, R2 = OMe, | NI till 2 | NI till 2 | NI till 2 |
| | | R = 3- | | | |
| | | thienyl | | | |
| 21 | 1FA | R1 = R2 = Cl, R = 4,5- | NI till 4 | NI till 4 | NI till 4 |
| | | dibromo-2- | | | |
| | | thienyl | | | |
| 22 | 1FB | R1 = Cl, R2 = H, | 1–2 | 2–4 | 1–2 |
| | | R = 4,5-dibromo- | | | |
| | | 2-thienyl | | | |
| 23 | 1FC | R1 = Br, R2 = H, | 1–2 | 2–4 | 1–2 |
| | | R = 4,5-dibromo- | | | |
| | | 2-thienyl | | | |
| 24 | FE | R1 = Br, R2 = OMe, | 4–8 | NI till 4 | NI till 4 |
| | | R = 4,5- | | | |
| | | dibromo-2-thienyl | | | |
| 25 | 1GA | R1 = R2 = Cl, R = —CH₂CH₂Cl | 4 | 2 | 2 |
| 26 | 1GB | R1 = Cl, R2 = H, | 2 | 2 | 4 |
| | | R = —CH₂CH₂Cl | | | |
| 27 | 1GC | R1 = Br, R2 = H, | 2 | 4 | 2 |
| | | R = —CH₂CH₂Cl | | | |
| 28 | 1GE | R1 = Br, R2 = OMe, | 4–8 | 4–8 | 8–16 |
| | | R = —CH₂CH₂Cl | | | |
| 29 | 1HA | R1 = R2 = Cl, R = —(CH₂)₃Cl | 1–2 | 1–2 | 1–2 |
| 30 | 1HB | R1 = Cl, R2 = H, | 1–2 | 2–4 | 1–2 |
| | | R = —(CH₂)₃Cl | | | |
| 31 | 1HC | R1 = Br, R2 = H, | 1–2 | 4–8 | 1–2 |
| | | R = —(CH₂)₃Cl | | | |
| 32 | 1HD | R1 = F, R2 = H, | 1–2 | 1–2 | 1–2 |
| | | R = —(CH₂)₃Cl | | | |
| 33 | 1HE | R1 = Br, R2 = OMe, | NI till 4 | NI till 4 | NI till 4 |
| | | R = —(CH₂)₃Cl | | | |
| 34 | 1IE | R1 = Br, R2 = OMe, | NI till 8 | NI till 8 | NI till 8 |
| | | R = —(CH₂)₄Cl | | | |
| 35 | 1JB | R1 = Cl, R2 = H, | NI till 2 | NI till 2 | NI till 2 |
| | | R = 3-iodophenyl | | | |
| 36 | 1JC | R1 = Br, R2 = H, | 2–4 | NI till 4 | NI till 4 |
| | | R = 3-iodophenyl | | | |
| 37 | 1JE | R1 = Br, R2 = OMe, | NI till 2 | NI till 2 | NI till 2 |
| | | R = 3- | | | |
| | | iodophenyl | | | |
| 38 | 1KB | R1 = Cl, R2 = H, | 0.5–1 | 1–2 | 1–2 |
| | | R = 2-pyrrolyl | | | |
| 39 | 1KC | R1 = Br, R2 = H, | 1–2 | 2–4 | 1–2 |
| | | R = 2-pyrrolyl | | | |

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

We claim:

1. A compound of formula (I) 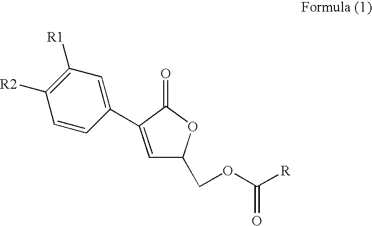
wherein R is substituted alkyl, alkenyl, aryl, heteroaryl, 2-thienyl, 3-thienyl, halothienyl, haloalkyl, halophenyl, or pyrrolyl; and R₁ and R₂, each independent of the other, is hydrogen, halogen, or alkoxy; with the proviso that R cannot be p-ClC₆H₄, CH₃ or (CH₃)₃C, when a) R₁ is H and R₂ is OCH₃, b) R₁ and R₂ are both Cl, c) R₁ is Cl and R₂ is H, or d) R₁ is H and R₂ is Br.

2. The compound of claim 1, wherein R is phenyl, allyl, 2-thienyl, 3-thienyl, 4,5-dibromo-2-thienyl, 2-chloroethyl, 3-chloropropyl, 4-chlorobutyl, 3-iodophenyl, or 2-pyrrolyl; and R₁ and R₂ are, idenpendently, H, Br, Cl, F, or methoxy.

3. A pharmaceutical composition comprising the compound of claim 1, its solvates, or its pharmaceutically acceptable salt having at least one salt forming group thereof; and a pharmaceutically acceptable diluent or carrier.

4. The pharmaceutical composition of claim 1 for the treatment or prevention of fungal infections.

5. A method for treating or preventing fungal infection in a subject, said method comprising administering a compound of claim 1, its pharmaceutically acceptable salt having at least one salt forming group, or its solvate to the subject.

6. The of claim 4, wherein the subject is animal or human being.

7. A process for the preparation of compound of formula (1) as claimed in claim 1, wherein R=substituted alkyl or alkenyl or aryl or heteroaryl or 2-thienyl or 3-thienyl or halothienyl or haloalkyl or halophenyl or pyrrolyl and the remaining radicals R₁ and R₂ each independently of others hydrogen or halogen or alkoxy, to claim 1 or its solvate or pharmaceutically acceptable salt of such compound having at aleast one salt forming group, said process comprising steps of:
a) contacting aldehyde of formula (2) 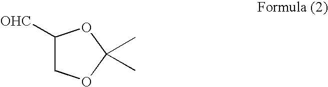
with phenyl acetates of formula (3) 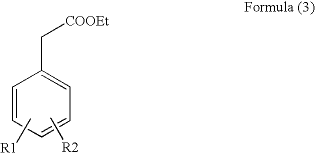
wherein R1 and R2 are independently H, Br, Cl, F and/or methoxy, in an organic solvent in presence of base to obtain compound of formula (4) 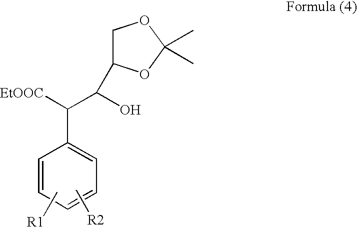
wherein R1 and R2 are independently H, Br, Cl, F, and/or methoxy;
b) contacting hydroxy ester of formula (4) with an acidic catalyst in an alcoholic solvent to obtain the alcohol of the formula (5), 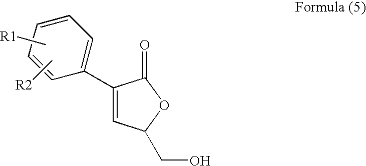
c) reacting the alcohol of the formula (5) with an acid chloride in an organic solvent in presence of a catalyst to obtain the compound of formula (1) 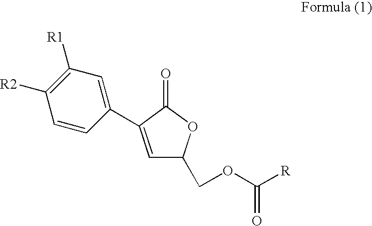
wherein R is substituted alkyl, alkenyl, aryl, heteroaryl, 2-thienyl, 3-thienyl, halothienyl, haloalkyl, halophenyl, or pyrrolyl; and R₁ and R₂, each independent of the other, are hydrogen, halogen, or alkoxy; and
d) converting the compound of formula (1) to its pharmaceutically acceptable salt by adapting conventional method.

8. The process of claim 7, wherein in step (a) the organic solvent used is selected from the group consisting of ethers, tetrahydrofuran, and diethyl ether.

9. The process of claim 7, wherein in step (a) the base is selected from the group consisting of alkyl lithiums, alkali metal hydrides, and alkali metal carbonates.

10. The process of claim 9, wherein in the base is n-butyllithium or sodium hydride.

11. The process of claim 7, wherein in step (b) the acidic catalyst used is selected from the group consisting of organic and inorganic acids.

12. The process of claim 11, wherein in the acidic catalyst is p-toluene sulfonic acid.

13. The process of claim 7, wherein in step (b) the solvent is selected from the group consisting of alcohols and hydrocarbons.

14. The process of claim 13, wherein the solvent is methanol or ethanol.

15. The process of claim 7, wherein in step (c) the catalyst is an organic base

16. The process of claim 15, wherein in the catalyst is pyridine.

17. The process of claim 7, wherein in step (c) the organic solvent is an alkyl halide.

18. The process of claim 17, wherein the organic solvent is dichloromethane or chloroform.

19. An compound of the formula (4) 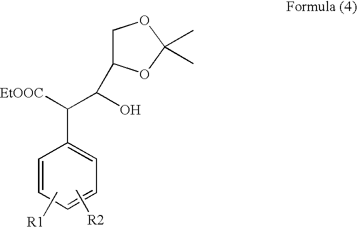
wherein R₁ and R₂ are each independently of others hydrogen or halogen or alkoxy.
